(19) United States
(12) Patent Application Publication
(10) Pub. No.: US 2022/0340935 A1
(43) Pub. Date: Oct. 27, 2022

(54) METHODS FOR CHOMOSOME REARRANGEMENT

(71) Applicant: APPLIED STEMCELL, INC., Milpitas, CA (US)

(72) Inventors: Qi ZHENG, San Ramon, CA (US); Ling-jie KONG, Union City, CA (US); Ruby Yanru TSAI, San Jose, CA (US)

(21) Appl. No.: 17/620,754

(22) PCT Filed: Jun. 19, 2020
(86) PCT No.: PCT/US2020/038578

§ 371 Date: Dec. 20, 2021

Related U.S. Application Data
(60) Provisional application No. 62/863,829, filed on Jun. 19, 2019.

(51) Int. Cl. C12N 15/90; C12N 15/11; C12N 9/22; C12N 15/62

(52) U.S. Cl. CPC C12N 15/907; C12N 15/11; C12N 9/22; C12N 15/62; C12N 2310/20; C12N 2800/80

(57) ABSTRACT

Provided herein are methods for producing a cell having chromosome translocation, deletion or reversion. In one embodiment, the method for producing a cell having chromosome translocation comprises introducing site-specific nucleases to the cell to create double strand breaks in a first and a second chromosome; generating an intermediate fusion chromosome comprising a first segment of the first chromosome, a selection region and the second segment of a second chromosome; and creating double strand breaks at sites that flank the selection region, thereby generating a fusion chromosome comprising at least part of the first segment of the first chromosome linked to at least part of the second segment of the second chromosome.

### CROSS-REFERENCE TO RELATED APPLICATIONS

This application claims priority to U.S. provisional patent application No. 62/863,829, filed Jun. 19, 2019, the disclosure of which is incorporated herein by reference.

### FIELD OF THE INVENTION

The present invention generally relates to genome editing. More specifically, the present invention relates to methods for producing cells having chromosome rearrangement, such as chromosome translocation or inversion.

### BACKGROUND

Genome editing, or genome engineering, allows human manipulation to insert, delete, modify or replace DNA in the genome of a living organism. Typically, genome editing methods use engineered nucleases, such as zinc finger nucleases (ZFNs), transcription activator-like effector-based nucleases (TALENs), and CRISPR/Cas system to create site-specific double-strand breads at desired location in the genome. The induced double-strand breaks are then repaired through non-homologous end-joining (NHEJ) or homologous recombination (HR), resulting DNA insertion, deletion, modification or replacement at the desired location in the genome.

Chromosome rearrangement is a type of mutation involving a change in the structure of the native chromosome, including deletion, duplication, translocation and inversion. It has been a challenge to generate a cell with desired chromosome rearrangement using the current genome editing technology. There is a need to develop new methods to create desired chromosome rearrangement.

### SUMMARY OF THE INVENTION

In one aspect, the present disclosure provides a method for producing a genomic modified cell. In one embodiment, the method comprises introducing into a cell: (1) a first site-specific nuclease targeting a first site in a first chromosome, said first site dividing the first chromosome into a first segment and a second segment of the first chromosome, (2) a second site-specific nuclease targeting a second site in a second chromosome, said second site dividing the second chromosome into a first segment and a second segment of the second chromosome, and (3) a donor DNA comprising sequentially: a 5′ homologous arm, a selection region, and a 3′ homologous arm, wherein the 5′ homologous arm is homologous to a region in the first segment of the first chromosome which flanks the first site and wherein the 3′ homologous arm is homologous to a region in the second segment of the second chromosome which flanks the second site. The method further comprises generating an intermediate cell comprising an intermediate fusion chromosome comprising sequentially: the first segment of the first chromosome, the selection region, and the second segment of the second chromosome. The method further comprises introducing into the intermediate cell a third and a fourth site-specific nuclease or a site-specific recombinase that target the third and the fourth sites, respectively, wherein the third and the fourth sites flank the selection region, thereby generating a cell comprising a fusion chromosome comprising at least part of the first segment of the first chromosome linked to at least part of the second segment of the second chromosome.

In another embodiment, the method comprises: introducing into a cell: (1) a first site-specific nuclease targeting a first site in a chromosome, (2) a second site-specific nuclease targeting a second site in the chromosome, wherein the first and second sites divide the chromosome sequentially into a first segment, a second segment and a third segment, (3) a donor DNA comprising sequentially: a 5′ homologous arm, a selection region, and a 3′ homologous arm, wherein the 5′ homologous arm is homologous to a region in the first segment which flanks the first site, and wherein the 3′ homologous arm is homologous to a region in the second segment which flanks the second site in reverse direction. The method further comprises generating an intermediate cell comprising an intermediate fusion chromosome comprising sequentially: the first segment, the selection region, and the second segment which reverts its orientation when compared to its orientation in the chromosome. The method further comprises introducing into the intermediate cell a third and a fourth site-specific nucleases or a site-specific recombinase that target a third and a fourth sites, wherein the third and the fourth sites flank the selection region, thereby generating a cell comprising a fusion chromosome comprising at least part of the first segment linked to at least part of the second segment, wherein the second segment reverts its orientation when compared to its orientation in the chromosome.

In certain embodiments, the cell is a mammalian cell.

In certain embodiments, any of the first, second, third and fourth site-specific nuclease is a zinc finger nuclease (ZFN), a transcription activator-like effector-based nuclease (TALEN), a CRISPR/Cas protein, or a meganuclease.

In certain embodiments, the selection region comprises a positive selectable marker, such as a puromycin resistance gene, a neomycin resistance gene, a hygromycin resistance gene a blasticidin S resistance gene, or a fluorescent gene.

In certain embodiments, the selection region further comprises a negative selectable marker, such as a thymidine kinase gene.

In certain embodiments, the site-specific recombinase is Cre recombinase, Flp recombinase or an integrase.

In another aspect, the present disclosure provides a genomic modified cell produced according to the method described herein.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates an exemplary method for generating a genomic modified cell having chromosome translocation.

FIG. 2 illustrates an exemplary method for generating a genomic modified cell having chromosome deletion.

FIG. 3 illustrates an exemplary method for generating a genomic modified cell having chromosome inversion.

FIG. 4 illustrates an exemplary embodiment of a donor DNA.

FIG. 5 illustrates the western blot results to detect the expression of EML4-ALK fusion protein in a genomic modified cell.

### DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

As used herein, the singular forms “a”, “an” and “the” include plural references unless the context clearly dictates otherwise.

It is noted that in this disclosure, terms such as “comprises”, “comprised”, “comprising”, “contains”, “containing” and the like are inclusive or open-ended and do not exclude additional, un-recited elements or method steps. Terms such as “consisting essentially of” and “consists essentially of” allow for the inclusion of additional ingredients or steps that do not materially affect the basic and novel characteristics of the claimed invention. The terms “consists of” and “consisting of” are close ended.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

The term “chromosome” used herein refers to a DNA molecule with part or all of the genetic material, or genome, of an organism. In mammals, a typical chromosome has several hundred million base pairs of DNA.

As used herein, “chromosome rearrangement” refers to a mutation or chromosome abnormality involving a change in the structure of the native chromosome. Typically, the mutation of a chromosome rearrangement event involves the change of a large piece of DNA sequence, at least several hundred kilo base pairs (Kb). As used herein, a piece of chromosome or a segment of chromosome involved in a genome engineering method for chromosome rearrangement refers to a continuous part of chromosome that has at least several hundred Kb DNA. Chromosome rearrangement may involve several different classes of events, such as deletions, duplication, inversions and translocation. In nature, chromosome rearrangement usually occurs when a chromosome or two chromosomes break at two different location, followed by a rejoining of the broken ends to produce a new chromosomal arrangement of genes, different from the gene order of the chromosomes before they were broken.

The terms “complementarity” or “complementary” are used in reference to polynucleotides (i.e., a sequence of nucleotides) related by the base-pairing rules. Complementarity may be “partial,” in which only some of the nucleic acids' bases are matched according to the base pairing rules (e.g., 5, 6, 7, 8, 9, 10 out of 10 being 50%, 60%, 70%, 80%, 90%, and 100% complementary), or there may be “complete” or “total” complementarity between the nucleic acids. The degree of complementarity between nucleic acid strands has significant effects on the efficiency and strength of their hybridization to one another.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, a “nuclease” is an enzyme capable of cleaving the phosphodiester bonds between the nucleotide subunits of nucleic acids. A “site-specific nuclease” refers to a nuclease whose functioning depends on a specific nucleotide sequence. Typically, a site-specific nuclease recognizes and binds to a specific nucleotide sequence and cuts a phosphodiester bond within the nucleotide sequence. In certain embodiments, the double-strand break is generated by site-specific cleavage using a site-specific nuclease. Examples of site-specific nucleases include, without limitation, zinc finger nucleases (ZFNs), transcriptional activator-like effector nucleases (TALENs), meganuclease and CRISPR (clustered regularly interspaced short palindromic repeats)-associated (Cas) nucleases.

A site-specific nuclease typically contains a DNA-binding domain and a DNA-cleavage domain. For example, a ZFN contains a DNA binding domain that typically contains between three and six individual zinc finger repeats and a nuclease domain that consists of the FokI restriction enzyme that is responsible for the cleavage of DNA. The DNA binding domain of ZFN can recognize between 9 and 18 base pairs. In the example of a TALEN, which contains a TALE domain and a DNA cleavage domain, the TALE domain contains a repeated highly conserved 33-34 amino acid sequence with the exception of the 12^{th} and 13^{th} amino acids, whose variation shows a strong correlation with specific nucleotide recognition. For another example, Cas9, a typical Cas nuclease, is composed of an N-terminal recognition domain and two endonuclease domains (RuvC domain and HNH domain) at the C-terminus.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

As used herein, the term “recombinase” or “site-specific recombinase” refers to a family of highly specialized enzymes that promote DNA rearrangement between specific target sites (Esposito D and Scocca J J, Nucleic Acids Research (1997) 25:3605-3614; Nunes-Duby SE et al, Nucleic Acids Research (1998) 26:391-406; Stark W M et al, Trends in Genetics (1992) 8:432-439). Virtually all site-specific recombinases can be categorized within one of two structurally and mechanistically distinct groups: the tyrosine (e.g., Cre, Flp, and the lambda integrase) or serine (e.g., phiC31 integrase, Bxb1 integrase, gamma-delta resolvase, Tn3 resolvase and Gin invertase) recombinases. Both recombinase families recognize target sites composed of two inversely repeated binding elements that flank a spacer sequence where DNA breakage and re-ligation occur. The recombination process requires concomitant binding of two recombinase monomers to each target site: two DNA-bound dimers (a tetramer) then join to form a synaptic complex, leading to crossover and strand exchange.

As used herein, a “selectable marker” refers a gene whose expression in cells allows the cells to be enriched or depleted under particular culture conditions. A selectable marker may be a foreign gene or a cellular gene which is not naturally expressed or such a gene which is naturally expressed, but at an inappropriate level, in the target cell populations. If the expression of the gene allows the cells to be enriched under particular conditions, the selectable marker is a “positive selectable marker.” Typically, a positive selectable marker is a gene that encodes for antibiotic resistance and selecting for those cells that express the selection marker comprises introducing antibiotic into the culture. In use, application of the antibiotic selectively kills or ablates cells that do not express the marker, leaving behind a population of cells purified or enriched in respect of those expressing the antibiotic resistance. Examples of a positive selectable marker include aminoglycoside phosphotransferase (neomycin resistance gene), puromycin-N-acetyl transferase (puromycin resistance gene), hygromycin resistance gene, and blasticidin S deaminase (blasticidin S resistance gene). Other examples of positive selectable marker include genes that can be used to select through cell sorting, e.g., fluorescent proteins and cell surface markers. Conversely, if the expression of the gene allows the cells to be depleted under particular culture condition, the selectable marker is a “negative selectable marker.” Examples of a negative selectable marker include thymidine kinase gene. In use, application of ganciclovir kills the cells with expression of thymidine kinase. Other examples of negative selectable markers include DT toxin, cell death genes, such as TRAIL, caspases and BCL2 family genes.

The term “sequentially” when used to describe two polynucleotide sequences means that the two sequences are not overlap, while the first sequence can either locate at the upstream (5′) or downstream (3′) of the second sequence.

The term “site” when used in the context of a chromosome or chromosome rearrangement refers to a specific nucleic acid sequence in the chromosome.

The term “subject” or “individual” or “animal” or “patient” as used herein refers to human or non-human animal, including a mammal or a primate, in need of diagnosis, prognosis, amelioration, prevention and/or treatment of a disease or disorder such as viral infection or tumor. Mammalian subjects include humans, domestic animals, farm animals, and zoo, sports, or pet animals such as dogs, cats, guinea pigs, rabbits, rats, mice, horses, swine, cows, bears, and so on.

The term “target” or “targeting,” when used in the context of a site-specific nuclease or a site-specific recombinase, means that the site-specific nuclease or the site-specific recombinase recognizes a specific nucleic acid sequence at a particular location in the genome or chromosome. In certain embodiments, the site-specific nuclease is a ZFN or a TALEN. In such case, a ZFN or TALEN targeting a specific site means that the ZFN or TALEN recognizes a specific nucleic acid sequence at the site. In certain embodiments, the site-specific nuclease is a CRISPR/Cas protein. In such case, a guide sequence (that is, gRNA) is designed to have complementarity to a specific nucleic acid sequence (that is, a target sequence) at the site, where hybridization between the target sequence and the guide RNA promotes the formation of a CRISPR complex.

Site-Specific Nuclease and Site-Specific Recombinase

The methods of the present disclosure relate to generating genome modified cells having rearranged chromosome using genome editing enzymes. In certain embodiments, genome editing enzymes include, without limitation, site-specific nucleases (e.g., Cas9, ZFN, TALEN and meganuclease) and site-specific recombinases (e.g., Cre, FLP, lamda integrase, phiC31 integrase, Bxb1 integrase, gamma-delta resolvase, Tn3 resolvase and Gin invertase).

CRISPR (Clustered Regularly Interspaced Short Palindromic Repeats)/Cas system was originally found as transcripts and other elements in the prokaryotic cells involved in the expression of or directing the activity of CRISPR-associated (“Cas”) genes, including sequences encoding a Cas nuclease that cleaves the nucleic acid sequence and generates double strand break (DSB), a guide sequence, a trans-activating CRISPR (tracr) sequence, a tracr-mate sequence, or other sequences and transcripts from a CRISPR locus. In eukaryotic cells, the CRISPR/Cas system comprises a CRISPR-associated nuclease and a small guide RNA. The target DNA sequence (the protospacer) contains a “protospacer-adjacent motif” (PAM), a short DNA sequence recognized by the particular Cas protein being used. In certain embodiments, the CRISPR system comprises CRISPR/Cas system of type I, type II, and type III, which comprises protein Cas3, Cas9 and Cas10, respectively.

The RNA-guided endonuclease Cas9 is a component of the type II CRISPR system widely utilized generate gene-specific knockouts in a variety of model systems. In one embodiment of the present disclosure, the CRISPR/Cas nuclease is a “sequence-specific nuclease”. Introduction of ectopic expression of Cas9 and a single guide RNA (gRNA) is sufficient to lead to the formation of double-strand breaks (DSBs) at a specific genomic region of interest, which leads to an indel via non-homologous end joining (NHEJ) pathway. Indels often result in frameshift mutations, except when the number of inserted/deleted nucleotides is a multiple of 3.

Along with Cas endonuclease, CRISPR experiments require the introduction of a guide RNA containing an approximately 15 to 30 base sequence specific to a target nucleic acid (e.g., DNA). A gRNA designed to target a genomic region of interest, for example, a particular exon encoding a functional domain of a protein, will generate a mutation in each gene that encodes the protein. The resulted modified genomic region may comprise one or more variants, each of which is different in the mutation. For example, the mutation will result in a modified genomic region with a desired modification, and/or a modified genomic region with an undesired modification. This approach has been widely utilized to generate gene-specific knockouts in a variety of model systems. In certain embodiments, a gRNA has a length of 15, 16, 17, 18, 19, 20, 21, 22, 23, 24, 25, 26, 27, 28, 29 or 30 nucleotides. gRNA can be delivered into a eukaryotic cell or a prokaryotic cell as RNA or by transfection with a vector (e.g., plasmid) having a gRNA-coding sequence operably linked to a promoter.

In certain embodiments, the Cas nuclease and the gRNA are derived from the same species. In certain embodiments, the Cas nuclease is derived from, for example, Staphylococcus aureus, Staphylococcus epidermidis, Staphylococcus sciuri, Pseudomonas aeruginosa, Enterococcus faecium, Enterococcus faecalis, Escherichia coli, Klebsiella pneumoniae, Streptococcus pneumoniae, Streptococcus pyrogenes, Lactobacillus bulgaricus, Streptococcus thermophilus Vibrio cholera, Achromobacter xylosoxidans, Burkholderia cepacia, Citrobacter diversus, Citrobacter freundii, Micrococcus leuteus, Proteus mirabilis, Proteus vulgaris, Staphylococcus lugdunegis, Salmonella typhi, Streptococcus Group A, Streptococcus Group B, S. marcescens, Enterobacter cloacae, Bacillus anthracis, Bordetella pertussis, Clostridium sp., Clostridium botulinum, Clostridium tetani, Corynebacterium diphtheria, Moraxalla (Brauhamella) catarrhalis, Shigella spp., Haemophilus influenza, Stenotrophomonas maltophili, Pseudomonas perolens, Pseuomonas fragi, Bacteroides fragilis, Fusobacterium sp. Veillonella sp., Yersinia pestis, and Yersinia pseudotuberculosis.

A gRNA can be designed using any known software in the art, such as Target Finder, E-CRISPR, CasFinder, and CRISPR Optimal Target Finder.

In certain embodiments, the composition described herein comprises a nucleic acid encoding the Cas nuclease or the gRNA, wherein the nucleic acid is contained in a vector. In some embodiments, the composition comprises Cas nuclease protein and a DNA encoding the gRNA. In some embodiments, the composition comprises a first nucleic acid encoding the Cas nuclease and a second nucleic acid encoding the gRNA, whereas the first and the second nucleic acids are contained in one vector. In some embodiment, the first and the second nucleic acids are contained in two separate vectors. In some embodiments, at least one vector is a viral vector. In certain embodiments, the vector is AAV vector.

A zinc finger nuclease (ZFN) is an artificial restriction enzyme generated by fusing a zinc finger DNA-binding domain to a DNA-cleavage domain. Zinc finger domain can be engineered to target specific desired DNA sequences, which directs the zinc finger nucleases to cleave the target DNA sequences. Typically, a zinc finger DNA-binding domain contains three to six individual zinc finger repeats and can recognize between 9 and 18 base pairs. Each zinc finger repeat typically includes approximately 30 amino acids and comprises a ββα-fold stabilized by a zinc ion. Adjacent zinc finger repeats arranged in tandem are joined together by linker sequences. Various strategies have been developed to engineer zinc finger domains to bind desired sequences, including both “modular assembly” and selection strategies that employ either phage display or cellular selection systems (Pabo CO et al., “Design and Selection of Novel Cys2His2 Zinc Finger Proteins” Annu. Rev. Biochem. (2001) 70:313-40). The most straightforward method to generate new zinc-finger DNA-binding domains is to combine smaller zinc-finger repeats of known specificity. The most common modular assembly process involves combining three separate zinc finger repeats that can each recognize a 3 base pair DNA sequence to generate a 3-finger array that can recognize a 9 base pair target site. Other procedures can utilize either 1-finger or 2-finger modules to generate zinc-finger arrays with six or more individual zinc finger repeats. Alternatively, selection methods have been used to generate zinc-finger DNA-binding domains capable of targeting desired sequences. Initial selection efforts utilized phage display to select proteins that bound a given DNA target from a large pool of partially randomized zinc-finger domains. More recent efforts have utilized yeast one-hybrid systems, bacterial one-hybrid and two-hybrid systems, and mammalian cells. A promising new method to select novel zinc-finger arrays utilizes a bacterial two-hybrid system that combines pre-selected pools of individual zinc finger repeats that were each selected to bind a given triplet and then utilizes a second round of selection to obtain 3-finger repeats capable of binding a desired 9-bp sequence (Maeder ML et al., “Rapid ‘open-source’ engineering of customized zinc-finger nucleases for highly efficient gene modification”. Mol. Cell (2008) 3:294-301). The non-specific cleavage domain from the type II restriction endonuclease FokI is typically used as the cleavage domain in ZFNs. This cleavage domain must dimerize in order to cleave DNA and thus a pair of ZFNs are required to target non-palindromic DNA sites. Standard ZFNs fuse the cleavage domain to the C-terminus of each zinc finger domain. In order to allow the two cleavage domains to dimerize and cleave DNA, the two individual ZFNs must bind opposite strands of DNA with their C-termini a certain distance apart. The most commonly used linker sequences between the zinc finger domain and the cleavage domain requires the 5′ edge of each binding site to be separated by 5 to 7 bp.

A transcription activator-like effector nuclease (TALEN) is an artificial restriction enzyme made by fusing a transcription activator-like effector (TALE) DNA-binding domain to a DNA cleavage domain (e.g., a nuclease domain), which can be engineered to cut specific sequences. TALEs are proteins that are secreted by Xanthomonas bacteria via their type III secretion system when they infect plants. TALE DNA-binding domain contains a repeated highly conserved 33-34 amino acid sequence with divergent 12th and 13th amino acids, which are highly variable and show a strong correlation with specific nucleotide recognition. The relationship between amino acid sequence and DNA recognition allows for the engineering of specific DNA-binding domains by selecting a combination of repeat segments containing the appropriate variable amino acids. The non-specific DNA cleavage domain from the end of the FokI endonuclease can be used to construct TALEN. The FokI domain functions as a dimer, requiring two constructs with unique DNA binding domains for sites in the target genome with proper orientation and spacing. See Boch J, Nature Biotechnology (2011) 29:135-6; Boch J, Science (2009) 326: 1509-12; Moscou M J and Bogdanove A J, Science (2009) 326 (5959): 1501; Juillerat A et al., Scientific Reports (2015) 5: 8150; Christian et al., Genetics (2010) 186: 757-61; Li et al., Nucleic Acids Research (2010) 39: 1-14.

Meganucleases are endodeoxyribonucleases characterized by a large recognition site (double-stranded DNA sequences of 12 to 40 base pairs). As a result, the site recognized by a meganuclease generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

Among meganucleases, the LAGLIDADG family of homing endonucleases has become a valuable tool for the study of genomes and genome engineering over the past fifteen years. Meganucleases are “molecular DNA scissors” that can be used to replace, eliminate or modify sequences in a highly targeted way. By modifying their recognition sequence through protein engineering, the targeted sequence can be changed. Meganucleases are used to modify all genome types, whether bacterial, plant or animal. They open up wide avenues for innovation, particularly in the field of human health, for example the elimination of viral genetic material or the “repair” of damaged genes using gene therapy.

Site-specific recombinases refer to a family of enzymes that mediate the site-specific recombination between specific DNA sequences recognized by the enzymes. Examples of site-specific recombinase include, without limitation, Cre recombinase, Flp recombinase, the lambda integrase, gamma-delta resolvase, Tn3 resolvase, Sin resolvase, Gin invertase, Hin invertase, Tn5044 resolvase, Tn3 transposase, sleeping beauty transposase, IS607 transposase, Bxb1 integrase, wBeta integrase, BL3 integrase, phiR4 integrase, A118 integrase, TG1 integrase, MR11 integrase, phi370 integrase, SPBc integrase, SV1 integrase, TP901-1 integrase, phiRV integrase, FC1 integrase, K38 integrase, phiBTI integrase and phiC31 integrase.

Methods for Genome Editing

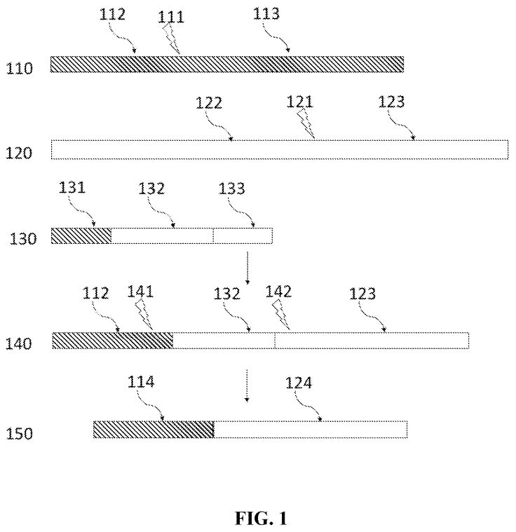

The present disclosure in one aspect provides methods for genome editing, such as chromosome translocation or inversion. The methods can be understood in the embodiments illustrated in FIGS. 1-3.

FIG. 1 illustrates an exemplary method for generating a genomic modified cell having chromosome translocation. Referring to FIG. 1, the method comprises introducing a cell at least a first site-specific nuclease, a second site-specific nuclease and a donor DNA 130. The first site-specific nuclease targets a site 111 in chromosome 110. The site 111 divides the chromosome 110 into a segment 112 and a segment 113. The second site-specific nuclease targets a site 121 in chromosome 120. The site 121 divides the chromosome 120 into a segment 122 and a segment 123. The donor DNA 130 includes a 5′ homologous arm 131, a selection region 132, and a 3′ homologous arm 133. The 5′ homologous arm 131 is homologous to a region in the segment 112 near the site 111. The 3′ homologous arm 133 is homologous to a region in the segment 123 near the site 121. The selection region 132 includes at least one positive selection marker.

As disclosed herein, the site-specific nuclease can be introduced into the cell using any means known in the art. In certain embodiments, the site-specific nuclease protein can be introduced into the cell. In certain embodiments, the site-specific nuclease can be expressed in the cell by inserting into the cell a nucleic acid sequence encoding the site-specific nuclease. The means of inserting the nucleic acid sequence into the cell include transfection, transformation, and transduction, wherein the nucleic acid sequence may be present in the cell transiently or may be incorporated into the genome of the cell (e.g., chromosome, plasmid, plastid, or mitochondrial DNA), converted into an autonomous replicon. Various techniques for transforming the nucleic acid sequence into the cell include, for example: microinjection, retrovirus mediated gene transfer, electroporation, transfection, or the like (see, e.g., Keown et al., Methods in Enzymology 1990, 185:527-537). In one embodiment, the nucleic acid sequence is introduced to the cell via a virus.

In certain embodiments, the first and the second site-specific nuclease is a CRISPR/Cas protein, e.g., a Cas9 protein. In such case, the method further comprises introducing to the cell a first guide RNA targeting the site 111 and a second guide RNA targeting the site 121.

The first and second site-specific nucleases, after being introduced into the cell, create double strand breaks at targeting site 111 and targeting site 121, respectively. The donor DNA 130 recombines with chromosome 110 and chromosome 120, generating an intermediate fusion chromosome 140, which comprises the segment 112, the selection region 132 and the segment 123. The presence of the positive selection marker in the selection region 132 allows selection of a cell comprising the intermediate fusion chromosome 140.

A third and a fourth site-specific nucleases are then introduced to the cell, generating double strand breaks at the site 141 in the segment 112 and the site 142 in the segment 123, respectively, wherein the site 141 and site 142 flank the selection region 132. As shown in FIG. 1, the targeting site 141 is located within the segment 112 and at or near the joining site between the segment 112 and the selection region. The targeting site 142 is located within the segment 123 and at or near the joining site between the segment 123 and the selection region. In certain embodiments, the targeting site 141 is about 0-10 kb (e.g., about 0 kb, 0.5 kb, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb or 10 kb) from the joining site between the segment 112 and the selection region. In certain embodiments, the targeting site 142 is about 0-10 kb (e.g., about 0 kb, 0.5 kb, 1 kb, 1.5 kb, 2 kb, 2.5 kb, 3 kb, 3.5 kb, 4 kb, 4.5 kb, 5 kb, 5.5 kb, 6 kb, 6.5 kb, 7 kb, 7.5 kb, 8 kb, 8.5 kb, 9 kb, 9.5 kb or 10 kb) from the joining site between the segment 123 and the selection region.

In certain embodiments, both the third and the fourth site-specific nucleases are a CRISPR/Cas protein, and two guide RNAs targeting the site 141 and site 142, respectively, are introduced to the cell, thereby generating the double strand breaks. In certain embodiments, two ZFNs or two TALENS targeting the site 141 and site 142, respectively, are introduced to the cell, thereby generating the double strand breaks. At least part of the segment 112 (segment 114) and at least part of the segment 123 (segment 124) that generated by the pair of site-specific nucleases then join through non-homologous end joining, generating a rearranged chromosome 150 comprising the segment 114 and the segment 124. In certain embodiments, the selection region 132 further comprises a negative selection marker, e.g. a thymidine kinase, which allows the selection of the rearranged chromosome 150, e.g., in the presence of ganciclovir.

In certain embodiments, each of the targeting site 141 and the targeting site 142 contains a recombinase recognition site, which can be introduced to the intermediate chromosome via the donor DNA 130. A site-specific recombinase, e.g., Cre recombinase, Flp recombinase and integrase, e.g., phiC31 integrase, is introduced to the cell to mediate recombination between the two recombinase recognition sites, generating the rearranged chromosome 150 comprising the segment 112 and the segment 123.

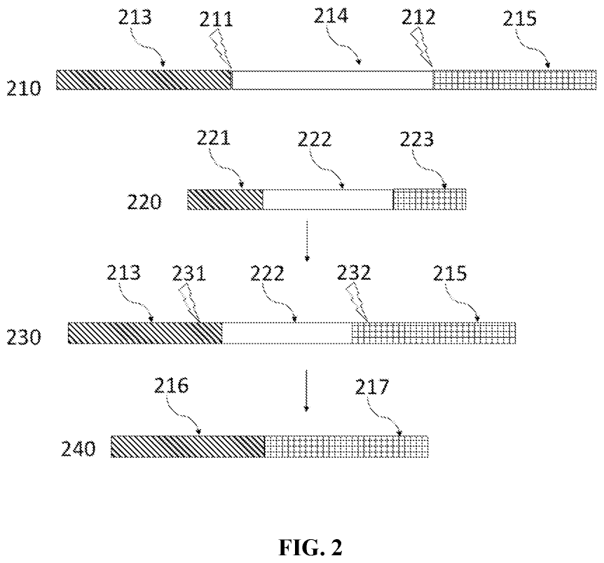

FIG. 2 illustrates an exemplary method for generating a genomic modified cell having a chromosome deletion. Referring to FIG. 2, the method comprises introducing a cell at least a first site-specific nuclease, a second site-specific nuclease and a donor DNA 220. The first site-specific nuclease targets the site 211 in chromosome 210. The second site-specific nuclease targets the site 212 in the chromosome 210. The sites 211 and 212 divide the chromosome 210 into a segment 213, a segment 214 and a segment 215. The donor DNA 220 includes a 5′ homologous arm 221, a selection region 222, and a 3′ homologous arm 223. The 5′ homologous arm 221 is homologous to a region in the segment 213 near the site 211. The 3′ homologous arm 223 is homologous to a region in the segment 215 near the site 212. The selection region 222 includes at least one positive selection marker.

The first and second site-specific nucleases, after being introduced into the cell, create double strand breaks at site 211 and site 212, respectively. The donor DNA 220 recombines with chromosome, generating an intermediate fusion chromosome 230, which comprises the segment 213, the selection region 222 and the segment 215. The presence of the positive selection marker in the selection region 222 allows selection of a cell comprising the intermediate fusion chromosome 230.

Like the embodiment illustrated in FIG. 1, the first and the second site-specific nuclease can be ZFNs or TALENs that target the site 211 and site 212. Alternatively, the first and the second site-specific nuclease is a CRISPR/Cas protein, and the method further comprises introducing into the cell two gRNA targeting the site 211 and the site 212, respectively.

A third and a fourth site-specific nucleases are then introduced to the cell, generating double strand breaks at the site 231 and the site 232, respectively, wherein the site 231 and site 232 flank the selection region 222. At least part of the segment 213 (segment 216) and at least part of the segment 215 (segment 217) generated by the pair of site-specific nucleases then join through non-homologous end joining, generating a rearranged chromosome 240 comprising the segment 216 and the segment 217. Alternatively, a site-specific recombinase is introduced to the cell, which mediate recombination between two recombinase recognition sites, near the site 231 and site 232 respectively, introduced to the intermediate chromosome via the donor DNA 220. In certain embodiments, the selection region 222 further comprises a negative selection marker, e.g. a thymidine kinase, which allows the selection of the rearranged chromosome 240, e.g., in the presence of ganciclovir.

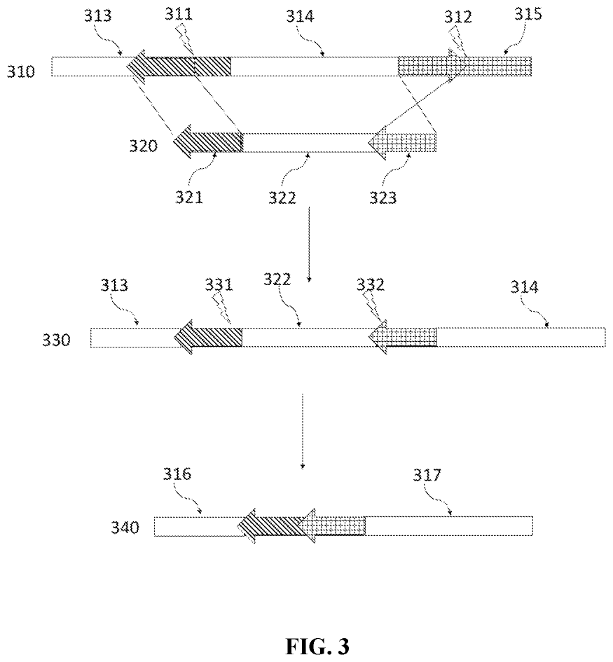

FIG. 3 illustrates an exemplary method for generating a genomic modified cell having chromosome inversion. Referring to FIG. 3, the method comprises introducing a cell at least a first site-specific nuclease, a second site-specific nuclease and a donor DNA 320. The first site-specific nuclease targets the site 311 in chromosome 310. The second site-specific nuclease targets the site 312 in chromosome 310. The sites 311 and 312 divide the chromosome 310 into a segment 313, a segment 314 and a segment 315. The sites 311 and 312 reside in a first gene and a second gene, respectively. The first gene and the second gene have opposite orientation in terms of gene expression. The donor DNA 320 includes a 5′ homologous arm 321, a selection region 322, and a 3′ homologous arm 323. The 5′ homologous arm 321 is homologous to a region in the segment 313 near site 311. The 3′ homologous arm 323 is homologous to a region in the segment 314 but has an opposite direction near the site 312. The selection region 322 includes at least one positive selection marker.

The first and second site-specific nucleases, after being introduced into the cell, create double strand breaks at site 311 and site 312, respectively. The donor DNA 320 recombines with chromosome 310, generating an intermediate fusion chromosome 330, which comprises the segment 313, the selection region 322 and the segment 314, wherein the orientation of the segment 314 is reversed relative to segment 313. The presence of the positive selection marker in the selection region 322 allows selection of a cell comprising the intermediate fusion chromosome 330.

A third and a fourth site-specific nucleases or a site-specific recombinase is then introduced to the cell, generating double strand breaks at the site 331 and site 332, respectively, wherein the site 331 and site 332 flank the selection region 322. At least part of segment 313 (segment 316) and at least part of the segment 314 (segment 317) then join through non-homologous end joining, generating a rearranged chromosome 340 comprising the segment 316 and the segment 317. In certain embodiments, the selection region 322 further comprises a negative selection marker, e.g. a thymidine kinase, which allows the selection of the rearranged chromosome 340, e.g., in the presence of ganciclovir.

The chromosome rearrangement also generates a fusion gene of the first gene and the second gene, wherein the orientation of the second gene is reversed.

The foregoing description of the invention has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. It is intended that the appended claims be construed to include other alternative embodiments of the invention; including equivalent components, methods, and means.

It is appreciated that the Summary and Abstract sections may set forth one or more, but not all exemplary embodiments of the present invention as contemplated by the inventor(s), and thus, are not intended to limit the present invention and the appended claims in any way.

### EXAMPLES

### Example 1

This example illustrates the production of a genome modified cell having a KIF5B-ALK translocation.

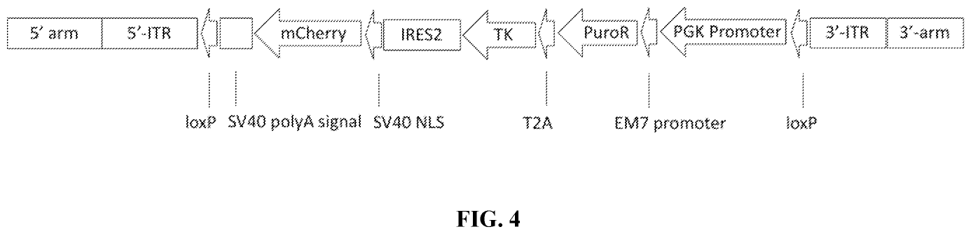

Methods: First, double stranded DNA break was introduced at KIF5B intron 24 and ALK intron 19, respectively, by an RNP method. A translocation donor including homologous arms of both genes, a puromycin-resist cassette and a TK cassette (FIG. 4) was co-transfected with RNA-protein complex. After puromycin selection, a KIF5B-Puro-TK-ALK fusion line was confirmed by genotyping result. The efficiency after the puromycin selection is about 1%. Second, the puromycin-resist gene and TK cassette were removed by using two new gRNAs. After GCV treatment, the single-cell clones were genotyped and 11.9% clones showed correct genotype.

Results: The inventors confirmed that the isolated cells have rearranged chromosome with KIF5B-ALK translocation. The inventor also confirmed the expression of KIF5B-ALK fusion mRNA in the isolated cells.

### Example 2

This example illustrates the stability of the production of a genome modified cell having a chromosome reversion resulting EML4-ALK fusion gene.

Methods: an RNP method was used to break EML4 intron 13 and ALK intron 19 simultaneously and inserted a transient puromycin-resist-TK selection cassette in the middle of the homologous arm bridge. This EML4-Puro-TK-ALK fusion line was then cut by a pair of gRNA/Cas9 protein complexes. The efficiency without GCV treatment is 13.5%.

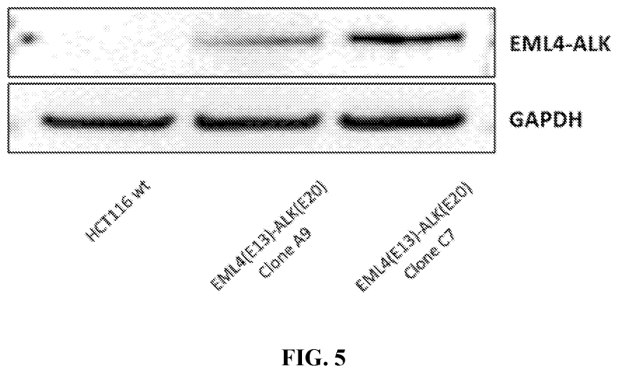

Results: The inventors confirmed that the isolated cells have rearranged chromosome with EML4-ALK fusion gene. The inventor also confirmed the expression of EML4-ALK fusion protein in the cell (see FIG. 5).

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A method for producing a genomic modified cell, the method comprising:
introducing into a cell:
a first site-specific nuclease targeting a first site in a first chromosome, said first site dividing the first chromosome into a first segment and a second segment of the first chromosome,
a second site-specific nuclease targeting a second site in a second chromosome, said second site dividing the second chromosome into a first segment and a second segment of the second chromosome, and
a donor DNA comprising sequentially:
a 5′ homologous arm,
a selection region, and
a 3′ homologous arm,
wherein the 5′ homologous arm is homologous to a region in the first segment of the first chromosome which flanks the first site and
wherein the 3′ homologous arm is homologous to a region in the second segment of the second chromosome which flanks the second site;
generating an intermediate cell comprising an intermediate fusion chromosome comprising sequentially:
the first segment of the first chromosome,
the selection region, and
the second segment of the second chromosome; and
introducing into the intermediate cell
(i) a third site-specific nuclease targeting a third site, and a fourth site-specific nuclease targeting a fourth site, wherein the third and the fourth sites flank the selection region, or
(ii) a site-specific recombinase targeting the third and the fourth sites, thereby generating a cell comprising a fusion chromosome comprising at least part of the first segment of the first chromosome linked to at least part of the second segment of the second chromosome.

2. The method of claim 1, wherein the cell is a mammalian cell.

3. The method of claim 1, wherein any of the site-specific nuclease is a zinc finger nuclease (ZFN), a transcription activator-like effector-based nuclease (TALEN), a meganuclease, or a CRISPR/Cas protein.

4. The method of claim 1, wherein the site-specific nuclease is a CRISPR/Cas protein, and the method further comprises introducing a first guide RNA targeting the first site in a first chromosome and a second guide RNA targeting the second site in a second chromosome.

5. The method of claim 1, wherein the donor DNA comprises a first and a second recombinase recognition site.

6. The method of claim 1, wherein the first chromosome is the same as the second chromosome.

7. The method of claim 1, wherein the first chromosome is different from the second chromosome.

8. The method of claim 1, wherein the selection region comprises a positive selectable marker.

9. The method of claim 8, wherein the positive selectable marker is a puromycin resistance gene, a neomycin resistance gene, a hygromycin resistance gene a blasticidin S resistance gene, or a fluorescent gene.

10. The method of claim 8, wherein the selection region further comprises a negative selectable marker.

11. The method of claim 10, wherein the negative selectable marker is a thymidine kinase gene.

12. The method of claim 1, wherein the site-specific nuclease is a CRISPR/Cas protein, and the method further comprising introducing a third guide RNA targeting the region near the third site and a fourth guide RNA targeting the region near the fourth site.

13. The method of claim 1, wherein the site-specific recombinase is Cre recombinase, Flp recombinase or an integrase.

14. A genomic modified cell produced according to the method of claim 1.

15. A method for producing a genomic modified cell, the method comprising:
introducing into a cell:
a first site-specific nuclease targeting a first site in a chromosome,
a second site-specific nuclease targeting a second site in the chromosome,
a donor DNA comprising sequentially:
a 5′ homologous arm,
a selection region, and
a 3′ homologous arm,
wherein the first and second sites divide the chromosome sequentially into a first segment, a second segment and a third segment,
wherein the 5′ homologous arm is homologous to a region in the first segment which flanks the first site, and
wherein the 3′ homologous arm is homologous to a region in the second segment which flanks the second site;
generating an intermediate cell comprising an intermediate fusion chromosome comprising sequentially:
the first segment,
the selection region, and
the second segment which reverts its orientation when compared to its orientation in the chromosome;
introducing into the intermediate cell
(i) a third site-specific nuclease targeting a third site, and a fourth site-specific nuclease targeting a fourth site, wherein the third and the fourth sites flank the selection region, or
(ii) a site-specific recombinase targeting the sites flanking the selection region, thereby generating a cell comprising a fusion chromosome comprising at least part of the first segment linked to at least part of the second segment, wherein the second segment reverts its orientation when compared to its orientation in the chromosome.

16. The method of claim 15, wherein the cell is a mammalian cell.

17. The method of claim 15, wherein any of the first, second, third and fourth site-specific nuclease is a zinc finger nuclease (ZFN), a transcription activator-like effector-based nuclease (TALEN), meganuclease, or a CRISPR/Cas protein.

18. The method of claim 15, wherein the first and the second site-specific nuclease is a CRISPR/Cas protein, and the method further comprises introducing a first guide RNA targeting the first site in a first chromosome and a second guide RNA targeting the second site in a second chromosome.

19. The method of claim 15, wherein the third and the fourth site-specific nuclease is a CRISPR/Cas protein, and the method further comprising introducing a third guide RNA targeting the region near the third site and a fourth guide RNA targeting the fourth site.

20. The method of claim 15, wherein the selection region comprises a positive selectable marker.

21. The method of claim 20, wherein the positive selectable marker is a puromycin resistance gene, a neomycin resistance gene, a hygromycin resistance gene a blasticidin S resistance gene, or a fluorescent gene.

22. The method of claim 20, wherein the selection region further comprises a negative selectable marker.

23. The method of claim 22, wherein the negative selectable marker is a thymidine kinase gene.

24. The method of claim 15, wherein the site-specific recombinase is Cre recombinase, Flp recombinase or an integrase.

25. The method of claim 15, wherein the third site resides in a first gene and divides the first gene into a first region and a second region of the first gene, wherein the fourth site resides in a second gene and divides the second gene into a first region and a second region of the second gene, wherein the fusion chromosome comprises a fusion gene comprises the first region of the first gene linked to the first region of the second gene, and wherein the first region of the second gene reverts its orientation compared to its orientation in the chromosome.

26. A genomic modified cell produced according to the method of claim 15.
